(19) United States
(12) Patent Application Publication
(10) Pub. No.: US 2020/0332245 A1
(43) Pub. Date: Oct. 22, 2020

(54) CONNECTION UNIT FOR CELL CULTURE DEVICE, INCUBATOR DEVICE, AND CELL CULTURE DEVICE

(71) Applicants: IHI CORPORATION, Tokyo (JP); Public University Corporation Yokohama City University, Yokohama-shi (JP)

(72) Inventors: Akihiko YOSHIMURA, Tokyo (JP); Kousuke ISHII, Tokyo (JP); Makiko SAITO, Tokyo (JP); Michinori SHIDA, Tokyo (JP); Kensuke HIRATA, Tokyo (JP); Shin-ichi SAKAI, Tokyo (JP); Yasuhiko FUKUCHI, Tokyo (JP); Hideki TANIGUCHI, Yokohama-shi (JP)

(73) Assignees: IHI CORPORATION, Tokyo (JP); Public University Corporation Yokohama City University, Yokohama-shi (JP)

(21) Appl. No.: 16/868,771

(22) Filed: May 7, 2020

Related U.S. Application Data
(63) Continuation-in-part of application No. PCT/JP2017/040282, filed on Nov. 8, 2017.

(51) Int. Cl. C12M 1/36; C12M 3/00; C12M 1/00; C12M 1/12

(52) U.S. Cl. CPC C12M 41/48; C12M 23/44; C12M 41/14; C12M 37/00; C12M 23/50

(57) ABSTRACT

A cell culture device includes an isolator and an incubator. A connection unit is used to connect the isolator and the incubator, and is provided with: a flexible cell supply tube extending from the incubator to the isolator; and a tube support unit having a holding part holding the cell supply tube and a connection end part attached to the incubator. The tube support unit can be switched between a first configuration in which the tube support unit supports the cell supply tube such that the cell supply tube extends in a first direction and a second configuration in which the tube support unit holds the cell supply tube such that the cell supply tube extends in a second direction. The second configuration is switched to the first configuration by means of the restoring force of the tube support unit.

### CROSS REFERENCE TO RELATED APPLICATIONS

This patent application is a continuation-in-part application claiming benefit under 35 U.S.C. § 365(c) to International Patent Application No. PCT/JP2017/040282 filed on Nov. 8, 2017. The International Application was published in Japanese on May 16, 2019, as International Publication No. WO 2019/092813 A1 under PCT Article 21(2).

### TECHNICAL FIELD

The present disclosure relates to a connection unit for a cell culture device, an incubator device, and a cell culture device.

### BACKGROUND ART

A cell culture device has an isolator and an incubator connected to the isolator.

The incubator is airtightly connected to the isolator via a door body. The door body of the incubator is opened and closed from the inside of the isolator. The internal space of the isolator is maintained in an aseptic state. Likewise, the internal space of the incubator is maintained in an aseptic state.

Accordingly, predetermined work in the incubator needs to be performed with a hand put in the incubator from the inside of the isolator via a glove fixedly provided in the isolator so that the aseptic state in the isolator is maintained.

Patent Literature 1 discloses a cell culture device. The cell culture device includes an incubator and an isolator. The incubator has a lid provided in a work chamber. The incubator is connected to the isolator. In addition, the isolator has a glove. The glove is provided toward the inside of the work chamber at a predetermined position of an openable and closable full door.

In addition, a device for automating cell culture has been proposed in the related art. Patent Literature 2 discloses a cell culture device. This device includes a culture tank, a culture solution tank, a gas supply tank, and the like. The tanks are interconnected by a supply pipe. According to this configuration, the cell culture device is capable of controlling the supply and discharge of a culture solution, a gas, and the like with respect to the culture tank.

Each of Patent Literature 3, Patent Literature 4, and Patent Literature 5 discloses a device related to cell culture. For example, the manufacturing device of Patent Literature 3 has an incubator accommodating a cell culture container and an isolator for treating cells sent from the incubator. The automatic culture device of Patent Literature 4 cultures a large amount of cells cultured and used for regenerative medicine for the human body. The cell culture system of Patent Literature 5 includes a container, an aseptic connector and an air filter connected to the container via a tube, and the like.

### CITATION LIST

### Patent Literature

Patent Literature 1: Japanese Unexamined Patent Publication No. 2009-225742

Patent Literature 2: Japanese Unexamined Patent Publication No. 2015-136330

Patent Literature 3: International Publication No. 2016/147897

Patent Literature 4: International Publication No. 2007/083465

Patent Literature 5: Japanese Unexamined Patent Publication No. 2015-122986

### SUMMARY OF INVENTION

### Technical Problem

In a cell culture device, various operations required for cell culture are performed in an isolator. In order to maintain an aseptic state, a worker is not allowed to directly touch a device or the like disposed in the isolator. Accordingly, the worker performs the operations by using a glove for work such as the glove disclosed in Patent Literature 1.

Accordingly, the range of the work that is performed by the worker is limited to the work range of the glove. Accordingly, improvement of workability via a glove in an isolator has been desired in this field.

In this regard, the present disclosure describes a connection unit for a cell culture device, an incubator device, and a cell culture device with which workability via a glove can be improved.

### Solution to Problem

The present disclosure relates to a connection unit for a cell culture device used in a cell culture device provided with an isolator having a glove and a first connection wall and accommodating a first container and an incubator connected to the isolator via a second connection wall facing the first connection wall and accommodating a second container. The connection unit includes: a flexible tube having one end connected to the first container and the other end connected to the second container and extending from the incubator to the isolator via holes respectively provided in the first connection wall and the second connection wall; and a tube support unit having a holding part holding the tube and a connection end part attached to the incubator. The tube support unit is switchable between a first configuration in which the tube is supported such that the tube extends in a first direction from the second connection wall toward the first connection wall and a second configuration in which the tube is held such that the tube extends in a second direction intersecting with the first direction. The second configuration is switched to the first configuration by means of a restoring force of the tube support unit.

### Effects of Invention

According to the present disclosure, workability via the glove in the cell culture device is improved.

### BRIEF DESCRIPTION OF DRAWINGS

FIG. 1 is a diagram illustrating components of a cell culture device and the connection relationship of the components.

FIG. 2 is a perspective view illustrating a state where an incubator and an isolator of the cell culture device according to a first embodiment are interconnected.

FIG. 3 is a perspective view illustrating a state where the incubator and the isolator of the cell culture device according to the first embodiment are separated from each other.

FIG. 4A is an exploded perspective view illustrating a second connection wall according to the first embodiment, and FIG. 4B is an arrow view of FIG. 4A.

FIG. 5 is an exploded cross-sectional view illustrating the interconnection structure of the incubator and the isolator at a time when a connection unit is in a first configuration.

FIG. 6 is a cross-sectional view illustrating the interconnection structure of the incubator and the isolator at a time when the connection unit is in a second configuration.

FIG. 7 is an enlarged cross-sectional view illustrating the interconnection structure of the incubator and the isolator at a time when the connection unit is in the first configuration.

FIG. 8 is an exploded perspective view illustrating a seal part according to a second embodiment of the present disclosure.

FIG. 9A is a front view illustrating a first seal member, and FIG. 9B is a front view illustrating a second seal member.

FIG. 10A and FIG. 10B are cross-sectional views illustrating attachment of the seal part according to the second embodiment.

FIG. 11 is a diagram illustrating components of a cell culture device according to Modification Example 1 and the connection relationship of the components.

### DESCRIPTION OF EMBODIMENTS

The present disclosure relates to a connection unit for a cell culture device used in a cell culture device provided with an isolator having a glove and a first connection wall and accommodating a first container and an incubator connected to the isolator via a second connection wall facing the first connection wall and accommodating a second container. The connection unit includes: a flexible tube having one end connected to the first container and the other end connected to the second container and extending from the incubator to the isolator via holes respectively provided in the first connection wall and the second connection wall; and a tube support unit having a holding part holding the tube and a connection end part attached to the incubator. The tube support unit is switchable between a first configuration in which the tube is supported such that the tube extends in a first direction from the second connection wall toward the first connection wall and a second configuration in which the tube is held such that the tube extends in a second direction intersecting with the first direction. The second configuration is switched to the first configuration by means of a restoring force of the tube support unit.

This connection unit includes the tube interconnecting the first container and the second container, and the tube is provided with the tube support unit. The tube support unit is switched from the second configuration to the first configuration by the restoring force. According to the first configuration, the tube is supported so as to extend in the first direction from the first connection wall toward the second connection wall. Then, the tube is capable of extending from the second connection wall of the incubator toward the inside of the isolator. In other words, the tube is inserted through the hole of the first connection wall of the isolator and further extends into the isolator. As a result, the tube can be brought close to the work range of the glove provided in the isolator. Accordingly, workability via the glove can be improved.

The connection unit for a cell culture device may further include a closing part closing a hole of the incubator and allowing the tube to be inserted through the closing part. The connection end part of the tube support unit may be attached to the closing part. According to this configuration, the connection unit can be easily attached to and detached from the incubator.

In the connection unit for a cell culture device, the tube support unit may be a coil spring and the tube may be inserted inside the coil spring. According to this configuration, the configuration of the connection unit can be simplified and switching from the second configuration to the first configuration can be reliably performed by means of the restoring force of the coil spring.

Another form of the present disclosure relates to an incubator device connected to an isolator having a glove and a first connection wall and accommodating a first container. The incubator device includes: an incubator connected to the isolator via a second connection wall facing the first connection wall and accommodating a second container; a flexible tube having one end connected to the first container and the other end connected to the second container and extending from the incubator to the isolator via holes respectively provided in the first connection wall and the second connection wall; and a tube support unit having a holding part holding the tube and a connection end part attached to the incubator. The tube support unit is switchable between a first configuration in which the tube is supported such that the tube extends in a first direction from the second connection wall toward the first connection wall and a second configuration in which the tube is held such that the tube extends in a second direction intersecting with the first direction. The second configuration is switched to the first configuration by means of a restoring force of the tube support unit. This incubator device includes the connection unit including the tube and the tube support unit described above. As a result, it is possible to bring the tube close to the work range of the glove provided in the isolator. Accordingly, workability via the glove can be improved.

A cell culture device that is yet another form of the present disclosure includes: an isolator having a glove and a first connection wall and accommodating a first container; an incubator connected to the isolator via a second connection wall facing the first connection wall and accommodating a second container; a flexible tube having one end connected to the first container and the other end connected to the second container and extending from the incubator to the isolator via holes respectively provided in the first connection wall and the second connection wall; and a tube support unit having a holding part holding the tube and a connection end part attached to the incubator. The tube support unit is switchable between a first configuration in which the tube is supported such that the tube extends in a first direction from the second connection wall toward the first connection wall and a second configuration in which the tube is held such that the tube extends in a second direction intersecting with the first direction. The second configuration is switched to the first configuration by means of a restoring force of the tube support unit. This cell culture device includes the connection unit including the tube and the tube support unit described above. As a result, it is possible to bring the tube close to the work range of the glove provided in the isolator. Accordingly, workability via the glove can be improved.

### First Embodiment

Hereinafter, embodiments of the present disclosure will be described in detail with reference to the drawings. It should be noted that the same or corresponding parts are denoted by the same reference numerals in the drawings. Duplicate description will be omitted.

### Connection Configuration of Cell Culture Device

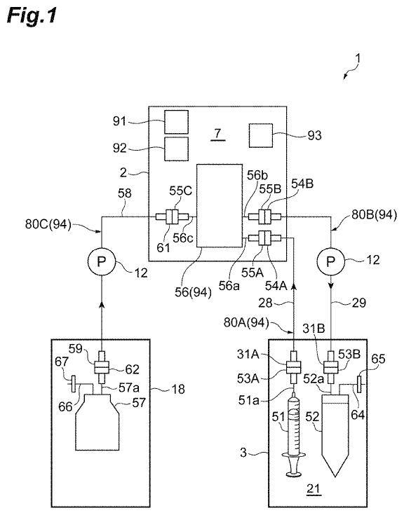

FIG. 1 is a diagram schematically illustrating components of a cell culture device 1 and the connection relationship of the components. As illustrated in FIG. 1, the cell culture device 1 has an incubator 2, an isolator 3, and a refrigerator 18.

The inside of the incubator 2 is a culture chamber 7. This culture chamber 7 accommodates a culture tank 56, an aeration tank 91, a heating tank 92, and a stripping tank 93. These tanks are interconnected by tubes. The culture tank 56 has three input-output ports. Specifically, the proximal ends of tubes 56a, 56b, and 56c are connected to the culture tank 56. An aseptic connector 55A is provided at the tip of the tube 56a. An aseptic connector 55B is provided at the tip of the tube 56b. An aseptic connector 55C is provided at the tip of the tube 56c.

The inside of the isolator 3 is a work chamber 21. The work chamber 21 accommodates cell supply means 51 and cell collection means 52. The cell supply means 51 and the cell collection means 52 may be collectively referred to as cell suction-discharge means. The proximal end of a tube 51a is connected to the cell supply means 51. An aseptic connector 53A is provided at the tip of the tube 51a. The proximal ends of tubes 52a and 64 are connected to the cell collection means 52. An aseptic connector 53B is provided at the tip of the tube 52a. In addition, the tube 64 is provided with an air filter 65.

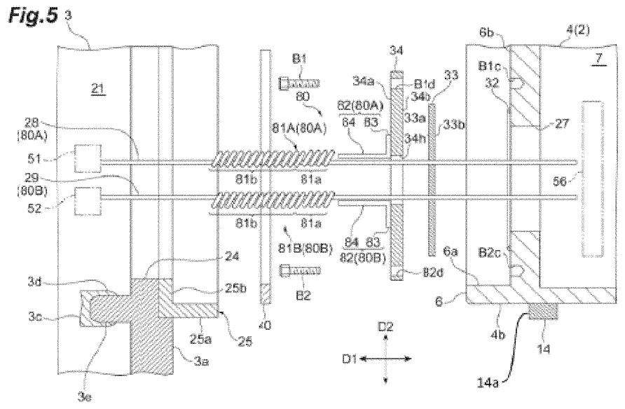

The culture tank 56 is connected to the cell supply means 51 by a first connection unit 80A. The first connection unit 80A has a cell supply tube 28, aseptic connectors 31A and 54A, and a first coil spring 81A (described later, see FIG. 5 and the like). In the first connection unit 80A, the aseptic connector 54A is connected to the aseptic connector 55A of the culture tank 56 and the aseptic connector 31A is connected to the aseptic connector 53A of the cell supply means 51.

Here, the culture tank 56, the cell supply means 51, and the first connection unit 80A constitute a closed system. Here, the “closed system” refers to a system having a closed space isolated from the outside world. Accordingly, the culture tank 56, the cell supply means 51, and the first connection unit 80A form a closed space. The inside of this closed space can be kept aseptic even after sterilization.

It should be noted that the aseptic connectors according to the present disclosure have a structure maintaining a closed state even when exposed to the outside. Accordingly, the aseptic connectors in the present disclosure are not limited to so-called commercially available aseptic connectors. For example, each of the aseptic connectors may have a structure in which a valve is opened and communication is achieved in the event of connection and the valve is airtightly closed in the event of removal. In addition, each of the aseptic connectors may be a tube having a structure in which a tip part can be closed by means of a pinch cock or the like in the event of removal.

The culture tank 56 is connected to the cell collection means 52 by a second connection unit 80B. The culture tank 56, the cell collection means 52, and the second connection unit 80B constitute a closed system. The second connection unit 80B has a cell collection tube 29, aseptic connectors 31B and 54B, and a second coil spring 81B (described later, see FIG. 5 and the like). In the second connection unit 80B, the aseptic connector 54B is connected to the aseptic connector 55B of the culture tank 56 and the aseptic connector 31B is connected to the aseptic connector 53B of the cell collection means 52. It should be noted that a pump device 12 is provided in the middle of the cell collection tube 29.

The refrigerator 18 accommodates a culture medium tank 57. The proximal ends of tubes 57a and 66 are connected to the culture medium tank 57. An aseptic connector 62 is provided at the tip of the tube 57a. The tube 66 is provided with an air filter 67. The culture medium tank 57 is connected to the culture tank 56 by a third connection unit 80C. The culture tank 56, the culture medium tank 57, and the third connection unit 80C constitute a closed system. The third connection unit 80C has a tube 58 and aseptic connectors 59 and 61. In the third connection unit 80C, the aseptic connector 59 is connected to the aseptic connector 62 of the culture medium tank 57 and the aseptic connector 61 is connected to the aseptic connector 55C of the culture tank 56. Further, the pump device 12 is provided in the middle of the tube 58 as in the case of the second connection unit 80B.

### Specific Configuration of Cell Culture Device

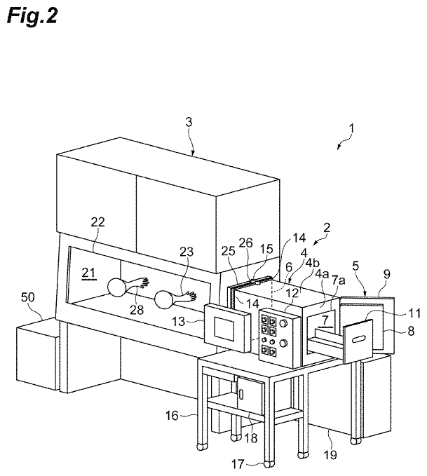

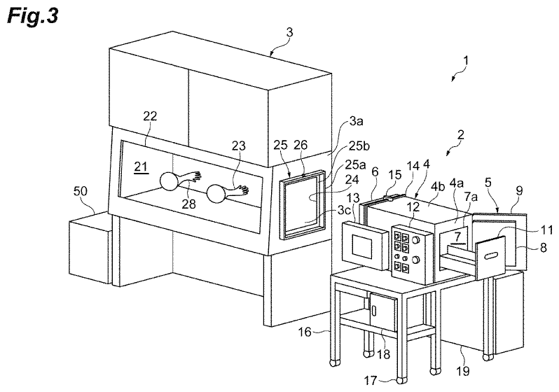

Next, a specific configuration of the cell culture device 1 will be described. The cell culture device 1 is disposed in, for example, a clean room. As illustrated in FIG. 2, the cell culture device 1 has the incubator 2 and the isolator 3. FIG. 2 is a perspective view illustrating the cell culture device 1 in a state where the incubator 2 and the isolator 3 are interconnected. FIG. 3 is a perspective view illustrating the cell culture device 1 in a state where the interconnection of the incubator 2 and the isolator 3 is released. It should be noted that the lower right side with respect to the paper surface of FIG. 2 is the forward direction in the following description. In addition, the upper left side with respect to the paper surface is the rearward direction in the following description.

The incubator 2 has an incubator main body 4 and a door body 5.

The incubator main body 4 is, for example, a rectangular box body. The incubator main body 4 has a front wall 4a, a side wall 4b, and a second connection wall 6. An opening 7a is provided in the front wall 4a. The incubator main body 4 forms the culture chamber 7. The culture chamber 7 is surrounded by the front wall 4a, the side wall 4b, and the second connection wall 6. The culture chamber 7 accommodates various devices required for cell culture treatment. Examples of the various devices include the culture tank 56 (see FIG. 1) for culturing cells. It should be noted that examples of the various devices may include a microscope for confirming a cell culture situation. In addition, the various devices may be interconnected via an aseptic connector. In addition, the various devices are isolated from the outside air by the incubator main body 4. Accordingly, the aseptic states inside the devices are maintained regardless of the state of the culture chamber 7.

The incubator main body 4 further has a drawer part 11 disposed in the culture chamber 7. The drawer part 11 is provided with various devices such as the culture tank 56. The drawer part 11 can be drawn out of the culture chamber 7. Work such as device installation and inter-device connection is performed outside the incubator 2 by the drawer part 11 being drawn out. In other words, the drawer part 11 is a worktable.

The door body 5 is provided on the front wall 4a of the incubator main body 4. In other words, the door body 5 is disposed on a surface on the side that is opposite to the second connection wall 6. Further, the door body 5 closes the opening 7a. The door body 5 keeps the inside of the incubator main body 4 (culture chamber 7) airtight. The door body 5 has a double structure. The door body 5 has an inner door 8 and an outer door 9. The inner door 8 is made of, for example, glass. The inner door 8 has the same size as the opening 7a or is larger in size than the opening 7a. With such a size, the inner door 8 switches between a configuration in which the opening 7a is closed and a configuration in which the opening 7a is opened. In addition, the outer door 9 keeps the culture chamber 7 airtight.

It should be noted that the inner door 8 of the door body 5 may be omitted in a case where the drawer part 11 is a configuration closing the opening 7a.

The incubator 2 further has the pump device 12 and a monitor 13. The pump device 12 and the monitor 13 are provided on a surface (such as the side wall 4b) different from the front wall 4a of the incubator main body 4 and the second connection wall 6.

The pump device 12 has various pumps. Examples of the various pumps include a liquid delivery pump for supplying a culture medium to the culture tank 56 (see FIG. 1). The pump device 12 supplies a culture medium and the like to the incubator 2. In addition, the pump device 12 adjusts the supply amount of the culture medium.

The monitor 13 is, for example, a touch panel-type liquid crystal display. The monitor 13 displays culture situation-related information. Examples of the culture situation-related information include an image of a culture situation. Such an image is acquired by a microscope installed in the culture chamber 7. In addition, control of the pump device 12 and the like are performed based on the culture situation-related information.

Further, the incubator 2 has an incubator-side frame part 14. The incubator-side frame part 14 is provided on the side wall 4b of the incubator main body 4. Specifically, the incubator-side frame part 14 is provided at the end part of the side wall 4b that is on the second connection wall 6 side. The incubator-side frame part 14 has a rectangular cross-sectional shape. The incubator main body 4 has an engagement member 15 such as a clamp. The engagement member 15 is provided on the upper and side surfaces of the incubator main body 4 and the incubator-side frame part 14. For example, the engagement member 15 is provided closer to the door body 5 side than the second connection wall 6 on the upper and side surfaces. The engagement member 15 is provided in, for example, three places (only one place is illustrated in FIGS. 2 and 3).

The incubator 2 is placed on a base 16 having four legs. A caster 17 is provided at the lower end of the leg via an adjuster for height adjustment (not illustrated). The base 16 can be moved on a floor surface by the caster 17. In other words, the incubator 2 is movable on a floor surface together with the base 16.

The refrigerator 18 is installed on the base 16. The refrigerator 18 accommodates the culture medium tank 57 (see FIG. 1) storing a culture medium. The tube 58 (see FIG. 1) is connected to the culture medium tank 57. The tube 58 is connected to a predetermined culture device (such as the culture tank 56) of the culture chamber 7 via the pump device 12. In the culture chamber 7, predetermined treatment is performed on the culture medium provided from the culture medium tank 57. Examples of the predetermined treatment include heating of the culture medium. After the treatment, the culture medium is supplied to the culture tank 56 (see FIG. 1).

A control panel 19 is installed on the base 16. The control panel 19 controls the operation of the pump device 12 and the like. This control is based on a control signal input from the monitor 13, a computer (not illustrated), or the like.

The isolator 3 has the work chamber 21 formed inside. A decontamination gas supply device 50 is connected to the isolator 3. The decontamination gas supply device 50 supplies a decontamination gas to the work chamber 21 of the isolator 3. Examples of the decontamination gas include hydrogen peroxide vapor. As a result, decontamination is performed in the work chamber 21 and the inside of the work chamber 21 becomes aseptic.

The isolator 3 has a window 22. The window 22 is a transparent wall that separates the work chamber 21 of the isolator 3 from the outside while making the work chamber 21 visible from the outside of the isolator 3. The window 22 is provided on a side surface. The window 22 is formed of a transparent glass plate, an acrylic plate, or the like. With the window 22, it is possible to confirm the intra-chamber state of the work chamber 21 from the outside. The window 22 is provided with gloves 23 attached toward the inside of the work chamber 21. The number of the gloves 23 is, for example, two. A worker wears the gloves 23 by inserting his or her hands into the gloves 23 from the outside. As a result, it is possible to perform work in the work chamber 21 while maintaining an aseptic state in the work chamber 21. It should be noted that the number of the gloves 23 is not limited to two. The number of the gloves 23 may be three or more.

As illustrated in FIG. 3, the isolator 3 has an opening 24. The opening 24 is provided in one surface (such as a first connection wall 3a). The isolator 3 has a door body 3c. The door body 3c switches the opening 24 between an open state and a closed state. In the closed state, the door body 3c airtightly closes the opening 24. The isolator 3 has a rectangular isolator-side frame part 25. The isolator-side frame part 25 is provided around the opening 24. The isolator-side frame part 25 has an L-shaped cross section. The isolator-side frame part 25 has a mating part 25a protruding forward and a flange part 25b formed on an inner edge.

The rear end part (second connection wall 6) of the incubator main body 4 is fitted into the isolator-side frame part 25. Accordingly, the isolator-side frame part 25 is larger than the rear end part of the incubator main body 4. Specifically, the isolator-side frame part 25 is larger than the second connection wall 6. An engaged member 26 is provided on the isolator-side frame part 25. The engaged member 26 engages with the engagement member 15. The engaged member 26 is provided in three predetermined places on a peripheral surface (only one place is illustrated in FIGS. 2 and 3).

### Seal Part

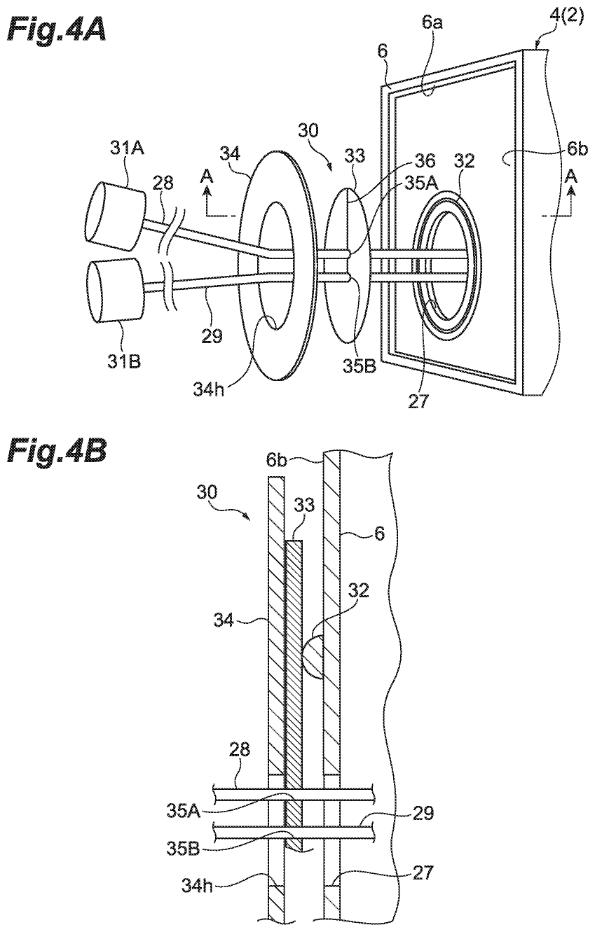

Next, a seal part 30 (closing part) will be described in detail with reference to FIG. 4A and FIG. 4B. It should be noted that the left side with respect to the paper surface is the tip and the right side with respect to the paper surface is the proximal end in FIG. 4A and FIG. 4B.

As illustrated in FIG. 4A, a recess part 6a is provided in the second connection wall 6 and the bottom of the recess part 6a is a bottom surface 6b. It should be noted that the second connection wall 6 may include the recess part 6a and the bottom surface 6b. A circular tube insertion hole 27 is provided in the bottom surface 6b. The tube insertion hole 27 allows the culture chamber 7 to communicate with the outside. The tube insertion hole 27 allows tubes to extend to the outside. The tubes are sterilized by a material-specific method such as autoclave or gamma ray sterilization. The tubes include the cell supply tube 28 and the cell collection tube 29.

The cell supply tube 28 and the cell collection tube 29 are cell suction-discharge lines. The cell suction-discharge lines are connected in a closed system to, for example, the culture tank 56 in the culture chamber 7. An aseptic connector is attached to end parts of the tubes. The aseptic connector 31A is attached to the tip of the cell supply tube 28. The aseptic connector 31B is attached to the tip of the cell collection tube 29.

The seal part 30 closes the tube insertion hole 27. The seal part 30 has a ring-shaped seal projection 32, a disk-shaped seal plate 33, and an annular pressing plate 34.

The seal projection 32 is an annular member surrounding the tube insertion hole 27. The seal projection 32 vertically protrudes from the bottom surface 6b of the second connection wall 6 over the entire circumference thereof. The seal projection 32 is, for example, an O-ring or a metallic ring protruding from the second connection wall 6. The seal projection 32 has a semicircular cross-sectional shape. It should be noted that the seal projection 32 may have any shape insofar as airtight contact is possible between the seal projection 32 and the seal plate 33. The seal projection 32 may be, for example, a ring having a triangular cross section.

The seal plate 33 is a circular and flexible film-shaped member and is pressed against the bottom surface 6b and the seal projection 32. The outer diameter of the seal plate 33 is, for example, larger than the outer diameter of the seal projection 32. In other words, the outer diameter of the seal plate 33 is larger than the inner diameter of the tube insertion hole 27. The seal plate 33 is made of, for example, silicone rubber. The seal plate 33 is capable of coming into contact with the seal projection 32 in an airtight manner.

The seal plate 33 has holes 35A and 35B. The holes 35A and 35B are provided substantially in the middle of the seal plate 33. The cell supply tube 28 is inserted through the hole 35A. The cell collection tube 29 is inserted through the hole 35B. The seal plate 33 has a cut 36. The cut 36 extends from an outer edge toward the holes 35A and 35B. The cell supply tube 28 is disposed in the hole 35A via the cut 36, and the cell collection tube 29 is disposed in the hole 35B. The places of connection between the holes 35A and 35B and the cut 36 are caulked. As a result of this caulking, airtightness can be ensured in the place of connection between the cell supply tube 28 and the seal plate 33 and the place of connection between the cell collection tube 29 and the seal plate 33.

It should be noted that the seal plate 33 may be a hard plate material such as a metal plate. In addition, at least one of the seal projection 32 and the seal plate 33 may be made of a material that can be elastically deformed as in the case of silicone rubber and satisfies predetermined requirements such as atmospheric resistance and chemical resistance.

The pressing plate 34 is an annular and hard plate material having a hole 34h and is pressed against the bottom surface 6b and the seal plate 33. The pressing plate 34 is fixed to the bottom surface 6b by screws B1 and B2 (see FIG. 5). The seal plate 33 is pressed against the seal projection 32 by the pressing plate 34 being fixed to the bottom surface 6b. Accordingly, the tube insertion hole 27 is airtightly closed by the seal projection 32 and the seal plate 33.

The inner diameter of the hole 34h of the pressing plate 34 is smaller than the outer diameter of the seal projection 32. The inner diameter of the hole 34h is smaller than the outer diameter of the seal plate 33. Further, the outer diameter of the pressing plate 34 is larger than the outer diameter of the seal plate 33.

### Structure of Incubator-Isolator Interconnection

The structure of interconnection of the incubator 2 and the isolator 3 will be described in detail below.

FIG. 5 is a cross-sectional view illustrating the interconnection structure of the incubator 2 and the isolator 3 in an enlarged and exploded manner. As illustrated in FIG. 5, the recess part 6a is provided in the second connection wall 6 of the incubator 2. The tube insertion hole 27 is provided in the bottom surface 6b of the recess part 6a. In addition, the incubator-side frame part 14 is disposed so as to surround the side wall 4b of the incubator main body 4. An end surface 14a of the incubator-side frame part 14 is shifted with respect to the second connection wall 6.

The tube insertion hole 27 of the incubator 2 is closed by the seal part 30. Holes B1d and B2d are provided in the pressing plate 34. The holes B1d and B2d are through holes. In addition, holes B1c and B2c are provided in the bottom surface 6b of the incubator main body 4. The holes B1c and B2c, which have bottoms, are not through holes. In addition, each of the holes B1c and B2c has an inner peripheral surface provided with a thread. Further, the screw B1 passes through the hole B1d and is screwed into the hole B1c. In addition, the screw B2 passes through the hole B2d and is screwed into the hole B2c. The seal part 30 is provided near the frame part that forms the lower side of the incubator-side frame part 14. In other words, the tube insertion hole 27 is provided near the frame part that forms the lower side of the incubator-side frame part 14.

The opening 24 is provided in the first connection wall 3a of the isolator 3. The isolator-side frame part 25 is provided so as to surround the opening 24. The isolator-side frame part 25 has the mating part 25a and the flange part 25b. The mating part 25a has a rectangular frame shape, and the second connection wall 6 is inserted into the region that is surrounded by this mating part 25a. Specifically, the end part of the incubator main body 4 that protrudes from the end surface 14a of the incubator-side frame part 14 and includes the second connection wall 6 is inserted into the region surrounded by the mating part 25a.

A sealing member 40 made of a soft resin such as an elastomer resin is provided inside the isolator-side frame part 25. With the second connection wall 6 of the incubator main body 4 inserted in the mating part 25a, the sealing member 40 is compressed between the second connection wall 6 and the flange part 25b. The opening 24 is airtightly closed by the compressed sealing member 40.

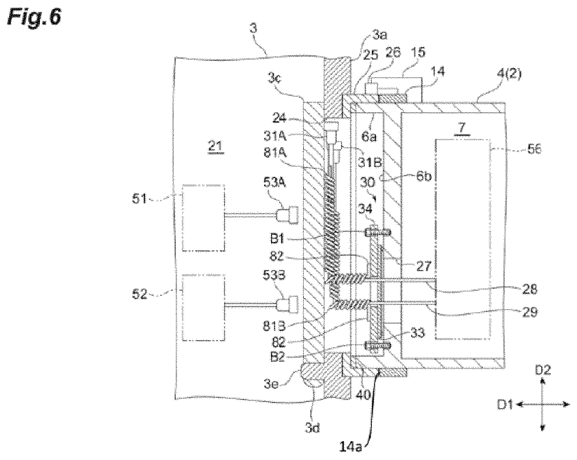

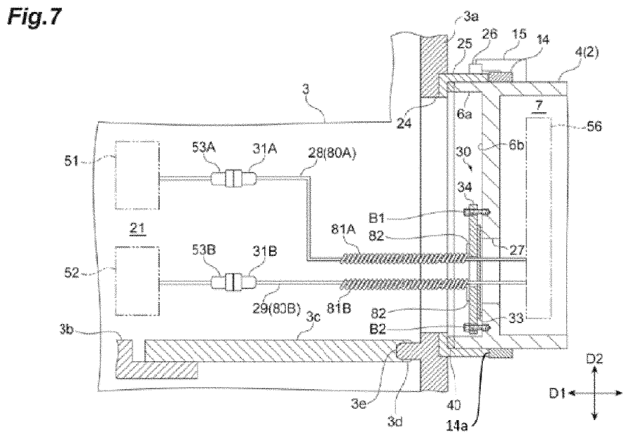

The door body 3c is connected to the inner wall surface of the first connection wall 3a of the isolator 3. The opening 24 is switched between an open configuration (see FIG. 7) and a closed configuration (see FIG. 6) by this door body 3c. The door body 3c is a plate-shaped member, and a lower end part 3d of the door body 3c is connected to a hinge 3e provided on the inner wall surface of the first connection wall 3a. Accordingly, the door body 3c opens the opening 24 by falling toward the inside of the isolator 3.

### Connection Unit for Cell Culture Device

A connection unit for a cell culture device (hereinafter, simply referred to as the connection unit 80) will be described in detail. The connection unit 80 includes the first connection unit 80A and the second connection unit 80B.

The first connection unit 80A interconnects the cell supply means 51 and the culture tank 56. The second connection unit 80B interconnects the cell collection means 52 and the culture tank 56. In addition, “interconnection” here means communication between the internal spaces of the cell supply means 51 and the cell collection means 52 and the internal space of the culture tank 56. Formed by this connection unit 80 is a closed space including the internal spaces of the cell supply means 51 and the cell collection means 52, the internal space of the cell supply tube 28, the internal space of the cell collection tube 29, and the internal space of the culture tank 56. This closed space is isolated from the culture chamber 7 of the incubator 2. In addition, the closed space is isolated from the work chamber 21 of the isolator 3. The cell supply means 51, the cell collection means 52, the culture tank 56, and the connection unit 80 forming the closed space as described above form a closed system. Further, the connection resulting in the formation of the closed space is referred to as hermetic connection.

Hereinafter, the first connection unit 80A will be described in detail. It should be noted that the second connection unit 80B will not be described in detail since the second connection unit 80B is similar in configuration to the first connection unit 80A. As described above, the first connection unit 80A includes the cell supply tube 28, the first coil spring 81A, and a tube support unit 82.

The cell supply tube 28 is inserted through the first coil spring 81A. The first coil spring 81A is attached to the proximal end of the cell supply tube 28 and supports the cell supply tube 28. Specifically, the first coil spring 81A is attached to the pressing plate 34 so as to extend in the normal direction of the second connection wall 6.

More specifically, the first coil spring 81A has a connection end part 81a and a holding part 81b. The connection end part 81a of the first coil spring 81A is inserted into the tube support unit 82 fixed to the pressing plate 34. In this configuration, the connection end part 81a of the first coil spring 81A is attached to the pressing plate 34 in the vertical direction. In other words, the position of the first coil spring 81A may be fixed in the vertical direction. The first coil spring 81A may or may not be fixed in the horizontal direction (direction in which the cell supply tube 28 extends). The tube support unit 82 has an L shape in cross section and has a proximal part 83 fixed to a surface 34a of the pressing plate 34 and a holding part 84 erect from the proximal part 83. The connection end part 81a of the first coil spring 81A is inserted into the holding part 84. In this configuration, the connection end part 81a of the first coil spring 81A is supported by the pressing plate 34. The cell supply tube 28 protrudes from the tip of the first coil spring 81A.

Here, the cell supply tube 28 is a flexible component having flexibility and low elasticity. In other words, the restoring force of the cell supply tube 28 attributable to deformation is smaller than the deforming force of the cell supply tube 28 attributable to gravity. Accordingly, the cell supply tube 28 bends vertically downward due to gravity when held such that the axis of the cell supply tube 28 becomes parallel with a horizontal axis.

Meanwhile, the first coil spring 81A is larger in elasticity than the cell supply tube 28. Accordingly, the first coil spring 81A exhibits a restoring force larger than the restoring force of the cell supply tube 28. Here, the restoring force refers to a force opposing the deforming force by which the first coil spring 81A is to bend due to gravity when the first coil spring 81A is held such that the axis of the first coil spring 81A becomes parallel with respect to the horizontal axis. Accordingly, in a case where the restoring force is large, the amount of deflection at the tip of the first coil spring 81A decreases. Conversely, in a case where the restoring force is small, the first coil spring 81A bends due to its own weight and the amount of deflection at the tip of the first coil spring 81A increases.

In other words, the first coil spring 81A increases the cross-sectional rigidity of the cell supply tube 28 bending due to its own weight and attempts to bring the extension direction of the cell supply tube 28 close to the horizontal direction. Then, the cell supply tube 28 protrudes to the inside from the outside of the isolator 3. In other words, the cell supply tube 28 approaches the work range of the glove 23. As a result, a worker can easily hold the cell supply tube 28 via the glove 23, and thus workability is improved.

### Cell Culture Method

Next, a method for culturing cells by using the cell culture device 1 will be described.

A step of preparing the first connection unit 80A is performed. First, a worker prepares the cell supply tube 28. Next, the worker inserts the cell supply tube 28 through the first coil spring 81A. Subsequently, the worker attaches the aseptic connector 31A to one end of the cell supply tube 28 and attaches the aseptic connector 54A to the other end of the cell supply tube 28. The first connection unit 80A is obtained as a result of this step.

In addition, the second connection unit 80B is prepared through a similar procedure.

Further, the third connection unit 80C is prepared. The third connection unit 80C has no coil spring whereas the first connection unit 80A has a coil spring. Accordingly, after preparing the tube 58, the worker attaches the aseptic connectors 59 and 61 to both ends of the tube 58, respectively.

Next, a step of sterilizing the connection unit 80 is performed. After the sterilization, the worker closes each aseptic connector. The aseptic state in the tube is maintained as a result of this action.

Next, a step of preparing an incubator device 94 is performed. Specifically, the worker prepares the culture tank 56. Next, the worker connects the connection unit 80 to the culture tank 56. Specifically, the aseptic connector 54A is connected to the aseptic connector 55A. Further, the aseptic connector 54B is connected to the aseptic connector 55B. Then, the aseptic connector 61 is connected to the aseptic connector 55C. Obtained as a result of this step is the incubator device 94 (see FIG. 1) having the culture tank 56, the first connection unit 80A, the second connection unit 80B, and the third connection unit 80C. These connections are performed with an aseptic state maintained.

Next, a step of removing the incubator 2 from the isolator 3 is performed.

Next, a step of accommodating a device in the incubator 2 is performed. Specifically, the worker opens the opening 7a by opening the door body 5 of the incubator 2. Next, the worker draws the drawer part 11 to the outside of the culture chamber 7 via the opening 7a. Then, outside the culture chamber 7, the worker installs the culture tank 56, a microscope or the like, and devices necessary for cell culture or observation in the drawer part 11.

It should be noted that the necessary devices installed here are, for example, the culture tank 56, the heating tank 92 heating a culture medium, a microscope, and the like. In addition, the aseptic connector 54A is already connected to the aseptic connector 55A of the culture tank 56. In other words, the culture tank 56, the aseptic connectors 54A and 55A, and the like are connected in advance as a unit (incubator device 94). In addition, the unit is a closed system. Further, the unit may be sterilized by means of an autoclave or the like after the unit becomes the closed system. The worker attaches the incubator device 94, which is a unit sterilized in this manner, to the drawer part 11.

Subsequently, the worker pushes the drawer part 11 into the culture chamber 7. As a result, the necessary devices are collectively stored in the culture chamber 7 together with the drawer part 11. It should be noted that the cell supply tube 28 and the cell collection tube 29 remain sterilized. Accordingly, the culture tank 56 remains aseptic even after the culture tank 56 is installed in the culture chamber 7. Next, the worker causes the aseptic connectors 53A, 53B, and 59 to extend from the tube insertion hole 27 to the outside of the culture chamber 7. In other words, among the configurations collectively stored in the culture chamber 7, the tip part (aseptic connector 53A) of the cell supply tube 28, the tip part (aseptic connector 53B) of the cell collection tube 29, and the tip part (aseptic connector 59) of the tube 58 are exposed to the outside of the culture chamber 7.

Next, a step of attaching the seal plate 33 to the first connection unit 80A and the second connection unit 80B is performed. Specifically, the worker disposes the cell supply tube 28 in the hole 35A via the cut 36 after disposing the cell collection tube 29 in the hole 35B via the cut 36. Next, the worker performs caulking treatment. Specifically, the worker joins the cut 36. In addition, the worker closes the gap between the hole 35A and the cell supply tube 28. Further, the worker closes the gap between the hole 35B and the cell collection tube 29. It should be noted that this work may be performed before the incubator device 94 is accommodated in the isolator 3 when the seal plate 33 is a flexible sheet member.

Next, a step of sandwiching the seal plate 33 between the pressing plate 34 and the bottom surface 6b of the second connection wall 6 is performed. Specifically, the worker presses the pressing plate 34 against the second connection wall 6 with the seal plate 33 in contact with the seal projection 32 and fixes the pressing plate 34 to the second connection wall 6 by using the screws B1 and B2. By the seal plate 33 being pressed by means of the pressing plate 34, the cell supply tube 28 and the cell collection tube 29 airtightly extend from the second connection wall 6.

Next, a step of accommodating devices in the isolator 3 is performed. Specifically, the worker disposes necessary devices in the work chamber 21 of the isolator 3. For example, the worker disposes the cell supply means 51 and the cell collection means 52 in the work chamber 21.

Next, a step of connecting the incubator 2 to the isolator 3 is performed. Specifically, the worker closes the opening 24. In other words, the worker presses the door body 3c of the isolator 3 against the inner wall surface of the first connection wall 3a. Next, the worker moves the base 16 such that the rear end part of the incubator main body 4 fits into the isolator-side frame part 25. At this time, a plurality of the engagement members 15 and a plurality of the engaged members 26 are used as marks for performing alignment. After the alignment is performed, the worker inserts the rear end part of the incubator main body 4 into the isolator-side frame part 25. Accordingly, the isolator-side frame part 25 functions as a guide at a time when the rear end part of the incubator main body 4 is inserted.

After inserting the rear end part of the incubator main body 4 into the isolator-side frame part 25, the plurality of engagement members 15 and the engaged members 26 are respectively engaged with each other by the worker. The incubator 2 and the isolator 3 are interconnected as a result of this engagement. At this time, the sealing member 40 (soft resin) in the isolator-side frame part 25 is compressed by the second connection wall 6 and the flange part 25b. Accordingly, airtightness is ensured at the part of connection between the incubator 2 and the isolator 3. In this state, the atmosphere in the culture chamber 7 and the atmosphere in the work chamber 21 are isolated from each other.

Here, as illustrated in FIG. 6, a space is formed between the incubator 2 and the isolator 3 when the incubator 2 is connected to the isolator 3. The cell supply tube 28 and the cell collection tube 29 are accommodated in the space. The lengths of the cell supply tube 28 and the cell collection tube 29 are longer than the length from the door body 3c to the bottom surface 6b. Accordingly, the cell supply tube 28 and the cell collection tube 29 are bent so as to be along a second direction D2 (up-down direction, left-right direction), which intersects with a first direction D1 from the incubator 2 toward the isolator 3. The cell supply tube 28 and the cell collection tube 29 have sufficient flexibility. Accordingly, the cell supply tube 28 and the cell collection tube 29 bend in the place of abutment against the door body 3c after, for example, extending from the incubator 2 toward the isolator 3 and then extend in the up-down or left-right direction along the door body 3c. In addition, the first coil spring 81A and the second coil spring 81B are attached to the proximal ends of the cell supply tube 28 and the cell collection tube 29, respectively. The first coil spring 81A and the second coil spring 81B also have flexibility, and thus the first coil spring 81A and the second coil spring 81B are deformed in accordance with the bending of the cell supply tube 28 and the cell collection tube 29.

The incubator 2 is connected to the isolator 3 in the first connection unit 80A and the second connection unit 80B in which the length of protrusion from the incubator 2 is longer than the distance from the door body 3c to the bottom surface 6b as described above. Then, the first connection unit 80A and the second connection unit 80B are accommodated, in a bent state, in the space formed between the incubator 2 and the isolator 3. This configuration of accommodation in a bent state is called a second configuration.

Here, the first coil spring 81A and the second coil spring 81B are larger in elasticity than the cell supply tube 28 and the cell collection tube 29. Accordingly, the first coil spring 81A and the second coil spring 81B in the second configuration have elastic energy converted into a restoring force. In other words, the first coil spring 81A and the second coil spring 81B in the second configuration generate a biasing force for pressing against the door body 3c. In other words, the door body 3c generates a reaction force opposing the biasing force.

Next, a step of opening the door body 3c of the isolator 3 is performed. Specifically, the worker opens the door body 3c by operating a handle driving the door body 3c or a switch of a motor driving the door body 3c. As illustrated in FIG. 7, the lower side of the door body 3c is connected to the hinge 3e. Accordingly, the upper side of the door body 3c falls toward the inside of the work chamber 21 of the isolator 3. Then, the door body 3c forms a part of the floor that defines the inside of the chamber of the isolator 3. Here, the door body 3c has generated a reaction force that bends the first coil spring 81A and the second coil spring 81B in the second direction D2. The reaction force is removed when the door body 3c is opened during this work. Then, the first coil spring 81A and the second coil spring 81B are returned to a first configuration by the restoring force. In other words, the first coil spring 81A and the second coil spring 81B return to the configuration (first configuration) of extension along the normal direction of the pressing plate 34. Further, the proximal end side of the cell supply tube 28 supported by the first coil spring 81A extends along the normal direction as a result of the return of the first coil spring 81A to the first configuration. In addition, the proximal end side of the cell collection tube 29 supported by the second coil spring 81B also extends along the normal direction. Accordingly, the cell supply tube 28 and the cell collection tube 29 extend toward the inside of the work chamber 21 to the extent that corresponds to the lengths of the first coil spring 81A and the second coil spring 81B. In other words, the work range of the glove 23 is approached.

Next, a step of decontaminating the inside of the incubator 2 and the isolator 3 is performed. Specifically, the worker supplies the work chamber 21 and the culture chamber 7 with a decontamination gas such as hydrogen peroxide vapor by operating the decontamination gas supply device 50. The inside of the work chamber 21 and the culture chamber 7 is decontaminated as a result of the decontamination gas supply. As a result, the inside of the work chamber 21 and the culture chamber 7 becomes aseptic.

Next, a step of connecting the aseptic connector 31A to the cell supply means 51 and connecting the aseptic connector 31B to the cell collection means 52 is performed. Specifically, the worker wears the glove 23. Next, the worker connects the aseptic connector 31A to the aseptic connector 53A and connects the aseptic connector 31B to the aseptic connector 53B in the work chamber 21. Here, as described above, the cell supply tube 28 provided with the aseptic connector 31A approaches the work range of the glove 23 without hanging down in the vicinity of the first connection wall 3a. Accordingly, the worker can easily hold the cell supply tube 28 via the glove 23.

Next, the culture medium tank 57 is connected to the culture tank 56. Specifically, the aseptic connector 59 is connected to the aseptic connector 62.

Preparations for culturing the cells are completed through the above steps. Next, a step of culturing the cells is performed.

The cell culture device 1 aseptically supplies the culture medium from the culture medium tank 57 into the culture chamber 7 via the third connection unit 80C by the pump device 12 being driven. As a result, the culture medium is finally supplied to the culture tank 56 through the aeration tank 91 and the heating tank 92. The gas concentration of the culture medium is adjusted in the aeration tank 91 such that the culture medium reaches an oxygen concentration and a pH suitable for cell culture. Further, the temperature of the culture medium is adjusted in the heating tank 92 such that the culture medium reaches a temperature suitable for cell culture. Accordingly, the culture medium supplied to the culture tank 56 is adjusted to an oxygen concentration, a pH, and a temperature suitable for cell culture.

In addition, in parallel with the culture medium supply, the cell culture device 1 aseptically supplies the cells or cells attached to a carrier or the like from the cell supply means 51 to the culture tank 56 via the first connection unit 80A. It should be noted that the cell supply by the cell supply means 51 is performed in the isolator 3 via the glove 23. In other words, the worker operates the cell supply means 51 by using the glove 23. Specifically, the worker holds a syringe that is the cell supply means 51 and pushes the piston of the syringe.

Culture treatment is initiated by the culture medium and the cells being supplied to the culture tank 56. The culture treatment is aseptically performed in the culture tank 56. Then, the worker drives the pump device 12 when it is determined that the culture treatment has been completed. By the pump device 12 being driven, the cells cultured in the culture tank 56 are collected from the culture tank 56 to the cell collection means 52 via the second connection unit 80B. It should be noted that the cells may be detached from the carrier, by passage through the stripping tank 93 in the process of collection from the culture tank 56 to the cell collection means 52, in the case of cell adhesion to the carrier.

It should be noted that the cell collection means 52 is provided in the isolator 3. Accordingly, any processing may be aseptically performed in the isolator 3 with respect to the cultured cells collected by the cell collection means 52.

With the cell collection completed, the worker releases the connection between the incubator 2 and the isolator 3. Specifically, the worker removes the aseptic connector 31A from the aseptic connector 53A and removes the aseptic connector 31B from the aseptic connector 53B. Next, the connection between the engagement member 15 and the engaged member 26 is released. Then, the incubator 2 is separated from the isolator 3 by the base 16 being moved.

It should be noted that the cell collection means 52 may be taken out of the work chamber 21 before the connection between the incubator 2 and the isolator 3 is released. Specifically, the cell collection means 52 is brought out of the work chamber 21 after the aseptic connector 53B is removed from the aseptic connector 31B. The cell collection means 52 is brought out in a predetermined collection place such as an aseptic chamber.

The worker removes the pressing plate 34 after releasing the connection between the incubator 2 and the isolator 3. Next, the worker pulls the cell supply tube 28 and the cell collection tube 29 out of the tube insertion hole 27.

When another treatment is subsequently performed, a step similar to the above-described procedure is performed. In other words, another cell supply tube 28 and another cell collection tube 29 are airtightly extended from the second connection wall 6. Next, the incubator 2 is connected to the isolator 3. Next, the aseptic connectors 31A and 31B are connected to the aseptic connectors 53A and 53B of the cell suction-discharge means in the aseptic work chamber 21. Next, cell suction-discharge treatment is performed. Next, the connection between the incubator 2 and the isolator 3 is released. Then, the cell collection means 52 is taken out.

In the cell culture device 1, the cell suction-discharge line having the cell supply tube 28 and the cell collection tube 29 is extended into the aseptic work chamber 21 as described above. Accordingly, work that is performed in the work chamber 21 is the connection and removal of the aseptic connector 31A and the aseptic connector 53A and the connection and removal of the aseptic connector 31B and the aseptic connector 53B that require work in an aseptic space.

Accordingly, work that is performed via the glove 23 can be only the minimum work (aseptic connector attachment and detachment) that needs to be performed in an aseptic space. Accordingly, there is no need to perform other work via the glove 23. Workability is improved as a result.

### Action and Effect

Hereinafter, the action and effect of the connection unit 80 will be described.

The connection unit 80 described above includes the cell supply tube 28 interconnecting the cell supply means 51 and the culture tank 56 and the cell collection tube 29 interconnecting the cell collection means 52 and the culture tank 56. The cell supply tube 28 is provided with the first coil spring 81A, and the cell collection tube 29 is provided with the second coil spring 81B. The first coil spring 81A and the second coil spring 81B are switched from the second configuration to the first configuration by the restoring force. According to the first configuration, the cell supply tube 28 and the cell collection tube 29 are supported so as to extend in the first direction D1 from the first connection wall 3a toward the second connection wall 6. Then, the cell supply tube 28 and the cell collection tube 29 are capable of extending from the second connection wall 6 of the incubator 2 toward the inside of the isolator 3. In other words, the cell supply tube 28 and the cell collection tube 29 are inserted through the opening 24 of the first connection wall 3a of the isolator 3 and further extend into the isolator 3. As a result, the cell supply tube 28 and the cell collection tube 29 can be brought close to the work range of the glove 23 provided in the isolator 3. Accordingly, workability via the glove 23 can be improved.

The connection unit 80 further has the seal part 30, which closes the tube insertion hole 27 of the incubator 2, and the cell supply tube 28 and the cell collection tube 29 are inserted through the seal part 30. The connection end part 81a of the second coil spring 81B and the first coil spring 81A is attached to the pressing plate 34 of the seal part 30. According to this configuration, the connection unit 80 can be easily attached to and detached from the incubator 2.

The connection unit 80 has the first coil spring 81A and the second coil spring 81B, which are tube support units. The cell supply tube 28 and the cell collection tube 29 are inserted into the first coil spring 81A and the second coil spring 81B. According to this configuration, the configuration of the connection unit 80 can be simplified and switching from the second configuration to the first configuration can be reliably performed by means of the restoring force of the first coil spring 81A and the second coil spring 81B.

The cell culture device 1 and the incubator device 94 having the connection unit 80 are capable of bringing a tube close to the work range of the glove 23 provided in the isolator. Accordingly, workability via the glove 23 can be improved.

By the way, in a cell culture device of the related art, a glove is fixedly provided in an isolator, and thus a work range is limited. In addition, the glove has a thickness of approximately several millimeters and work needs to be performed with another glove worn inside. As a result, it is difficult to perform detailed work with the cell culture device of the related art.

Further, in the case of the cell culture device of the related art, work that needs to be performed in an aseptic space includes treatment such as cell supply and collection. Accordingly, work that does not need to be performed in an aseptic state is also performed via the glove. Accordingly, the related art is poor in workability.

For example, a culture tank, a gas supply tank, and the like are installed in an incubator in a case where the cell culture device of Patent Literature 2 is installed in the incubator. Subsequently, the culture tank, the gas supply tank, and the like are interconnected by a supply pipe. Such work also needs to be performed via a glove. This results in poor workability.

In this regard, the cell culture device 1 according to the present disclosure has the incubator 2 including the culture chamber 7 formed inside, the opening 7a opening the culture chamber 7 to the outside, the second connection wall 6 as a surface different from the surface provided with the opening 7a, the tube insertion hole 27 formed in the second connection wall 6, the seal part 30 closing the tube insertion hole 27, and the cell suction-discharge line airtightly extending from the seal part 30. An aseptic connector is provided at the end part of the cell suction-discharge line that is hermetically connected to the culture tank 56 of the closed system.

The cell culture device 1 according to the present disclosure has the seal part 30. The seal part 30 has the seal projection 32 formed around the tube insertion hole 27, the seal plate 33 airtightly penetrated by the cell suction-discharge line and capable of coming into contact with the seal projection 32, and the pressing plate 34 pressing the seal plate 33 against the seal projection 32.

The cell culture device 1 according to the present disclosure further has the isolator 3 in which the work chamber 21 is formed. The isolator 3 has the opening 24 that can be airtightly closed in the first connection wall 3a.

The cell culture device 1 according to the present disclosure has the isolator-side frame part 25 provided around the opening 24. The incubator 2 and the isolator 3 are attached to and detached from the isolator-side frame part 25 by the second connection wall 6 being inserted and removed.

The method for interconnecting the incubator 2 and the isolator 3 according to the present disclosure has a step of installing the culture tank 56 in the culture chamber 7 of the incubator 2 from the opening 7a formed in the incubator 2, a step of extending the cell suction-discharge line extending from the culture tank 56 from the tube insertion hole 27 formed in the second connection wall 6 as a surface different from the surface provided with the opening 7a, a step of airtightly closing the tube insertion hole 27 by means of the seal part 30 airtightly penetrated by the cell suction-discharge line, and a step of interconnecting the incubator 2 and the isolator 3 such that the opening 24 formed in the first connection wall 3a of the isolator 3 is airtightly closed by the second connection wall 6.

In the method for interconnecting the incubator 2 and the isolator 3 according to the present disclosure, the cell culture device 1 has the incubator 2 having the culture chamber 7 formed in the incubator 2, the opening 7a opening to the outside from the culture chamber 7, the second connection wall 6 as a surface different from the surface provided with the opening 7a, the tube insertion hole 27 formed in the second connection wall 6, the seal part 30 closing the tube insertion hole 27, and the cell suction-discharge line airtightly extending from the seal part 30. Then, the aseptic connectors 31A and 31B are provided at the end part of the cell suction-discharge line that is hermetically connected to the culture tank 56 of the closed system. Accordingly, device installation in the culture chamber 7 via the opening 7a can be performed outside the incubator 2. Workability can be improved as a result.

The method for interconnecting the incubator 2 and the isolator 3 according to the present disclosure has a step of installing the culture tank 56 in the culture chamber 7 of the incubator 2 from the opening 7a formed in the incubator 2, a step of extending the cell suction-discharge line extending from the culture tank 56 from the tube insertion hole 27 formed in the second connection wall 6 provided on a surface different from the surface provided with the opening 7a, a step of airtightly closing the tube insertion hole 27 by means of the seal part 30 airtightly penetrated by the cell suction-discharge line, and a step of interconnecting the incubator 2 and the isolator 3 such that the opening 24 formed in the first connection wall 3a of the isolator 3 is airtightly closed by the second connection wall 6. Accordingly, the opening 24 can be airtightly closed with the isolator 3 and the incubator 2 isolated from each other. Further, it is possible to perform work in the culture chamber 7 while maintaining the aseptic state of the isolator 3.

The second connection wall 6 faces the front wall 4a where the door body 5 of the incubator 2 is provided. The second connection wall 6 is capable of airtightly closing the opening 24 by the incubator 2 being connected to the isolator 3. In other words, the incubator 2 is connected to the isolator 3 in a state where the atmosphere in the work chamber 21 and the atmosphere in the culture chamber 7 are isolated from each other.

Accordingly, the aseptic state in the work chamber 21 can be maintained regardless of the state in the culture chamber 7. Then, work that does not need to be performed in an aseptic space, such as installation of the culture tank 56, the culture medium tank 57, and the like and attachment of each tube to the pump device 12, can be performed in the culture chamber 7. Further, this work can be performed with the inner door 8 and the outer door 9 open. Workability is improved as a result.

The cell collection means 52 accommodating cells can be taken into and out of the culture chamber 7 with the culture tank 56 installed in the culture chamber 7. Accordingly, the amount of work required for cell supply and discharge can be reduced.

The drawer part 11 is provided in the culture chamber 7. Various devices such as the culture tank 56 and a microscope are installed on the drawer part 11. Then, the drawer part 11 functions as a worktable. Accordingly, it is possible to perform the work of the culture chamber 7 in an external space by pulling out the drawer part 11. Workability can be further improved as a result.

### Second Embodiment

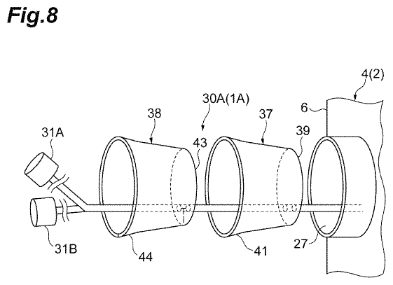

Next, a second embodiment of the present disclosure will be described with reference to FIGS. 8, 9, and 10. In addition, in FIG. 8, the left side with respect to the paper surface is the tip and the right side with respect to the paper surface is the proximal end.

As illustrated in FIG. 8, a cell culture device 1A has a seal part 30A. The seal part 30A airtightly closes the tube insertion hole 27. The seal part 30A has, for example, a second seal member 38 and a first seal member 37 made of silicone rubber.

The proximal end side of the first seal member 37 is closed by a first closing surface 39. The first seal member 37 is tubular and has an open tip side. The outer shape of the first seal member 37 is a truncated cone shape increasing in diameter toward the tip side. A first insertion part 41 having an increasing inclination and an increased diameter is formed at the tip part of the first seal member 37.

It should be noted that the outer diameter of the first closing surface 39 is smaller than the inner diameter of the tube insertion hole 27. The outer diameter of the first insertion part 41 is larger than the inner diameter of the tube insertion hole 27.

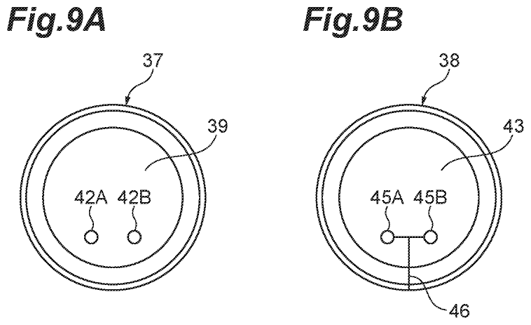

As illustrated in FIG. 9A, in addition, holes 42A and 42B are formed in the first closing surface 39. The cell suction-discharge line passes through the holes 42A and 42B. In other words, the hole 42A allows penetration by the cell supply tube 28. The hole 42B allows penetration by the cell collection tube 29. In other words, the cell supply tube 28 and the cell collection tube 29 airtightly penetrate the first closing surface 39 via the holes 42A and 42B.

The airtightness between the first closing surface 39 and the cell supply tube 28 and the airtightness between the first closing surface 39 and the cell collection tube 29 may be ensured by a step of inserting the cell supply tube 28 and the cell collection tube 29 into the holes 42A and 42B and a step of caulking the holes 42A and 42B being performed.

It should be noted that the airtightness between the first closing surface 39 and the cell supply tube 28 and the airtightness between the first closing surface 39 and the cell collection tube 29 may be ensured by either a step of forming a cut along a generatrix from the tip part to the proximal end part or a step of forming a cut radially reaching the holes 42A and 42B from a generatrix, a step of fitting the cell supply tube 28 and the cell collection tube 29 into the holes 42A and 42B from the cut, and a step of caulking the holes 42A and 42B and the cut being performed.

As illustrated in FIG. 9B, the second seal member 38 has the same shape as the first seal member 37. A second closing surface 43 is formed on the proximal end side of the second seal member 38. A second insertion part 44 is formed at the tip part of the second seal member 38. Holes 45A and 45B are formed in the second closing surface 43. The hole 45A allows penetration by the cell supply tube 28. The hole 45B allows penetration by the cell collection tube 29.

The second seal member 38 has a cut 46. The cut 46 extends along a generatrix from the tip part to the proximal end part. In addition, the cut 46 radially extends from the generatrix to the holes 45A and 45B. The cell supply tube 28 and the cell collection tube 29 are respectively fitted into the holes 45A and 45B via the cut 46.

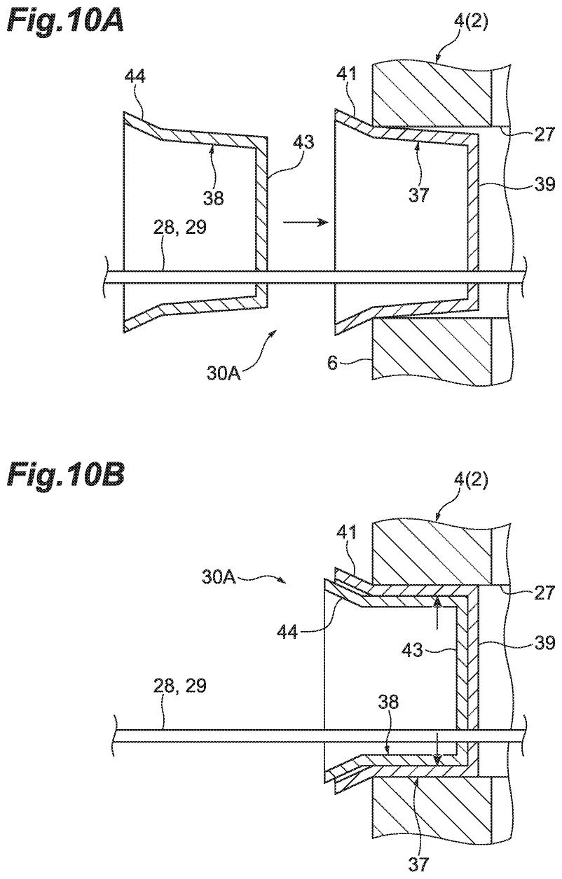

Next, a method for closing the tube insertion hole 27 by means of the seal part 30A will be described with reference to FIG. 10A and FIG. 10B.

First, the first seal member 37 is inserted into the tube insertion hole 27. At this time, the first seal member 37 is airtightly penetrated by the cell supply tube 28 and the cell collection tube 29. Then, the first seal member 37 is inserted from the first closing surface 39 side to the proximal end part of the first insertion part 41.

Next, the second seal member 38 is slid to the proximal end side along the cell supply tube 28 and the cell collection tube 29. The second seal member 38 is penetrated by the cell supply tube 28 and the cell collection tube 29. As a result, the second seal member 38 is inserted into the first seal member 37 via the first insertion part 41.

The second seal member 38 generates a restoring force when the second seal member 38 is inserted into the first seal member 37. This restoring force presses the first seal member 37 radially outward. In addition, as illustrated in FIG. 10B, the tube insertion hole 27 is airtightly closed by the first seal member 37 by the first seal member 37 being pressed against the tube insertion hole 27.

Further, the work chamber 21 is higher in pressure than the culture chamber 7 when the incubator 2 and the isolator 3 are connected to each other. Accordingly, the pressing force of the first seal member 37 with respect to the tube insertion hole 27 further increases. Airtightness can be further improved as a result.

As described above, in the second embodiment, the seal part 30A has the second seal member 38 and the first seal member 37 made of silicone rubber. Accordingly, the tube insertion hole 27 is airtightly closed by the first seal member 37 and the second seal member 38.

Accordingly, it is possible to perform work in the culture chamber 7 with the aseptic state in the work chamber 21 maintained. Further, work that is performed in the work chamber 21 is minimized. Workability is improved as a result.

The closing of the tube insertion hole 27 has a step of inserting the first seal member 37 into the tube insertion hole 27 and a step of inserting the second seal member 38 into the first seal member 37. Accordingly, connection by means of a bolt or the like does not have to be performed. The number of components can be reduced as a result. Further, connection can be facilitated. Workability is improved as a result.

### Modification Example

The connection unit 80, the incubator device 94, and the cell culture device 1 according to the present disclosure have been described in detail. However, the content of the present disclosure is not limited to the embodiments described above. Various modifications can be made to the content of the present disclosure within the gist of the present disclosure.

### Modification Example 1

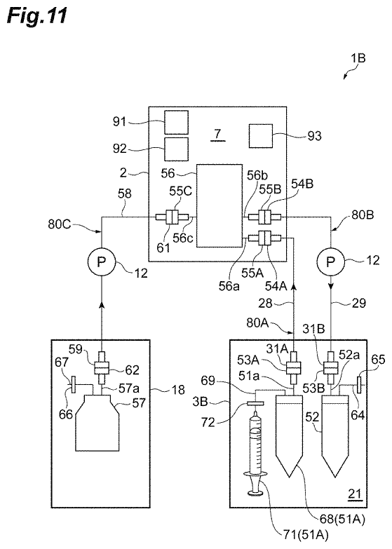

For example, cell supply means 51A may have a vial 68 and a syringe 71 connected to the vial 68 via a tube 69 as illustrated in FIG. 11. Cells are enclosed in the vial 68. Air is supplied into the vial 68 via the tube 69 by the syringe 71. As a result, the internal pressure of the vial 68 increases. Then, the cells in the vial 68 are pushed out to the culture tank 56. It should be noted that the tube 69 is provided with an air filter 72. According to this configuration, the aseptic state in the vial 68 is maintained even in the event of air supply from the syringe 71.

### Modification Example 2

The cell collection means according to Modification Example 2 may be similar in configuration to the cell supply means 51. In this configuration, the cell collection means has a vial accommodating collected cells and a syringe for suctioning out the cells. A worker depressurizes the inside of the vial by pulling the piston of the syringe. The cells in the culture tank 56 are suctioned out by the inside of the vial being depressurized. In this configuration, no pump device may be installed in the cell collection tube.

### Modification Example 3

One aseptic connector 31A and one aseptic connector 31B are attached to the tips of the cell supply tube 28 and the cell collection tube 29, respectively. For example, a plurality of aseptic connectors may be provided at the tip of each of the cell supply tube and the cell collection tube. In other words, the tips of the cell supply tube and the cell collection tube may branch into a plurality of tips with an aseptic connector attached to each of the branching tips. According to this configuration, a plurality of aseptic connectors are attached to the tip of the cell supply tube and a plurality of aseptic connectors are attached to the tip of the cell collection tube. As a result, it is possible to select an aseptic connector to be used based on the content of treatment. Accordingly, another treatment can be performed, with the incubator 2 and the isolator 3 interconnected, without the incubator 2 and the isolator 3 being disconnected each time treatment is completed. Workability can be further improved as a result.

### Modification Example 4

A method for closing the tube insertion hole 27 may have a step of attaching the seal plate 33 in advance to the cell supply tube 28 and the cell collection tube 29, a step of accommodating the cell supply tube 28, the cell collection tube 29, and the seal plate 33 in the culture chamber 7, a step of bending the seal plate 33 and taking one end part of the cell supply tube 28, one end part of the cell collection tube 29, and the seal plate 33 out of the tube insertion hole 27, and a step of closing the tube insertion hole 27. In this case, the seal plate 33 needs to be formed of a soft material such as silicone rubber.

### Modification Example 5

A method for closing the tube insertion hole 27 may have a step of providing the aseptic connector 31A at the proximal end of the cell supply tube 28, a step of providing the aseptic connector 31B at the proximal end of the cell collection tube 29, a step of closing the inside of the cell supply tube 28 and the cell collection tube 29, and a step of attaching the seal plate 33 to the cell supply tube 28 and the cell collection tube 29.

### Modification Example 6

A method for closing the tube insertion hole 27 may have a step of attaching the seal plate 33 to the cell supply tube 28 and the cell collection tube 29, a step of inserting the aseptic connectors 54A and 54B from the tube insertion hole 27 into the culture chamber 7, a step of interconnecting the aseptic connectors 54A and 54B and the aseptic connectors 55A and 55B of the culture tank 56 assembled in an aseptic state, and a step of closing the tube insertion hole 27. In this case, a hard material such as a metal plate can be used as the seal plate 33.

### Modification Example 7

The drawer part 11 of the incubator 2 may be omitted in a case where, for example, the depth of the culture chamber 7 is small and work in the culture chamber 7 can be easily performed.

The isolator 3 has been exemplified as an object of connection to the incubator 2. The object of connection to the incubator 2 may be another device that can be connected via the aseptic connectors 31A and 31B and requires work in the culture chamber 7 separately from the connection object of the aseptic connectors 31A and 31B. The object of connection to the incubator 2 may be, for example, a device having an aseptic space such as a safety cabinet and a clean bench. In this case, a structure attaching the incubator 2 to the back or side surface of the safety cabinet or the clean bench is provided and both are connected.

### Modification Example 8

In the seal part 30 of the present disclosure, the seal projection 32 protrudes from the bottom surface 6b and the seal projection 32 and the seal plate 33 are in airtight contact with each other. The seal projection for ensuring airtightness is not limited to the configuration that is provided on the second connection wall 6. For example, the seal projection may be provided on the seal plate. In this case, the seal projection protrudes from the seal plate toward the second connection wall. In addition, the seal projection may be provided on one or both of the second connection wall and the pressing plate. In this case, the seal part ensures airtightness between the second connection wall and the pressing plate. Further, the seal projection may be provided on one or both of the seal plate and the pressing plate. In this case, the seal part ensures airtightness between the seal plate and the pressing plate.

### Modification Example 9

A coil spring has been exemplified as the tube support unit of the present disclosure. The tube support unit may be any unit that has flexibility allowing bending when an external force is applied (second configuration) and exhibits a restoring force allowing a return to the original shape at a time of release from the external force (first configuration). For example, the tube support unit may be a torsion spring having one end fixed to the tube and the other end fixed to the pressing plate.

Furthermore, the device and method of the present disclosure may have the following configurations.

### Modification Example 10

Another form of the present disclosure relates to a cell culture device comprising an incubator having: a culture chamber formed inside; a first opening part opening outward from the culture chamber; a connection wall part of a surface different from the first opening part; an insertion hole formed in the connection wall part; a seal part blocking the insertion hole; a cell suction-discharge line airtightly extending from the seal part; and a drawer part provided so as to be insertable and removable with respect to the culture chamber, wherein an aseptic connector is provided at an extension end of the cell suction-discharge line hermetically connected to a culture tank of a closed system, and the drawer part is a worktable outside the incubator.

### Modification Example 11

In the cell culture device, the seal part has a seal projection formed around the insertion hole, a seal plate airtightly penetrated by the cell suction-discharge line and capable of coming into contact with the seal projection, and a pressing plate pressing the seal plate against the seal projection.

### Modification Example 12

In the cell culture device, the seal part has: a tubular first seal member having a proximal end side blocked on a first blocking surface airtightly penetrated by the cell suction-discharge line and increased in diameter toward a tip side; and a second seal member having a proximal end side blocked on a second blocking surface slidably penetrated by the cell suction-discharge line and increased in diameter toward a tip side, the first seal member is inserted from the proximal end side into the insertion hole, and the second seal member is inserted from the proximal end side into the first seal member.

### Modification Example 13

The cell culture device may further include an isolator in which a work chamber is formed, wherein the isolator has a second opening part in one surface and the connection wall part is capable of airtightly blocking the second opening part.

### Modification Example 14

In the cell culture device, a frame part is formed around the second opening part and the incubator and the isolator are attached and detached by the connection part being inserted into and removed from the frame part.

### Modification Example 15

Another form of the present disclosure relates to a connection method comprising: a step of drawing out a drawer part insertable and removable with respect to a culture chamber in an incubator from a first opening part formed in the incubator and installing a culture tank in the drawer part outside the incubator; a step of extending a cell suction-discharge line extending from the culture tank from an insertion hole formed in a connection wall part of a surface different from the first opening part; a step of airtightly blocking the insertion hole with a seal part airtightly penetrated by the cell suction-discharge line; and a step of interconnecting the incubator and an isolator such that a second opening part formed in one surface of the isolator is airtightly blocked by the connection wall part.

### REFERENCE SIGNS LIST

1, 1A: cell culture device, 2: incubator, 3: isolator, 3a: first connection wall, 3c, 5: door body, 3d: lower end part, 3e: hinge, 4: incubator main body, 4a: front wall, 4b: side wall, 6: second connection wall, 6a: recess part, 6b: bottom surface, 7: culture chamber, 7a: opening, 8: inner door, 9: outer door, 11: drawer part, 12: pump device, 13: monitor, 14: incubator-side frame part, 14a: end surface, 15: engagement member, 16: base, 17: caster, 18: refrigerator, 19: control panel, 21: work chamber, 22: window, 23: glove, 24: opening, 25: isolator-side frame part, 25a: fitting part, 25b: flange part, 26: engaged member, 27: tube insertion hole, 28: cell supply tube (tube), 29: cell collection tube (tube), 30, 30A: seal part (closing part), 31A, 31B, 53, 53A, 53B, 54A, 54B, 55A, 55B, 55C, 59, 61, 62: aseptic connector, 32: seal projection, 33: seal plate, 34: pressing plate, 34a: surface, 34h, 35A, 35B, 42A, 42B, 45A, 45B, B1c, B1d, B2c, B2d: hole, 36: cut, 37: first seal member, 38: second seal member, 39: first closing surface, 40: sealing member, 41: first insertion part, 43: second closing surface, 44: second insertion part, 46: cut, 50: decontamination gas supply device, 51: cell supply means (first container), 51A: cell supply means (first container), 51a, 52a: tube, 52: cell collection means (first container), 56: culture tank (second container), 56a, 56b, 56c, 57a, 58, 64, 66, 69: tube, 57: culture medium tank, 65, 67, 72: air filter, 68: vial, 71: syringe, 80: connection unit, 80A: first connection unit, 80B: second connection unit, 80C: third connection unit, 81a: connection end part, 81b: holding part, 82: tube support unit, 83: proximal part, 84: holding part, 91: aeration tank, 92: heating tank, 93: stripping tank, 94: incubator device, B1, B2: screw, D1: first direction, D2: second direction.

1. A connection unit for a cell culture device used in a cell culture device provided with an isolator having a glove and a first connection wall and accommodating a first container and an incubator connected to the isolator via a second connection wall facing the first connection wall and accommodating a second container, the connection unit comprising:
a flexible tube having one end connected to the first container and the other end connected to the second container and extending from the incubator to the isolator via holes respectively provided in the first connection wall and the second connection wall; and
a tube support unit having a holding part holding the tube and a connection end part attached to the incubator, wherein
the tube support unit is switchable between a first configuration in which the tube is supported such that the tube extends in a first direction from the second connection wall toward the first connection wall and a second configuration in which the tube is held such that the tube extends in a second direction intersecting with the first direction, and
the second configuration is switched to the first configuration by means of a restoring force of the tube support unit.

2. The connection unit for a cell culture device according to claim 1, further comprising a closing part closing a hole of the incubator and allowing the tube to be inserted through the closing part,
wherein the connection end part of the tube support unit is attached to the closing part.

3. The connection unit for a cell culture device according to claim 1, wherein
the tube support unit is a coil spring, and
the tube is inserted inside the coil spring.

4. An incubator device connected to an isolator having a glove and a first connection wall and accommodating a first container, the incubator device comprising:
an incubator connected to the isolator via a second connection wall facing the first connection wall and accommodating a second container;
a flexible tube having one end connected to the first container and the other end connected to the second container and extending from the incubator to the isolator via holes respectively provided in the first connection wall and the second connection wall; and
a tube support unit having a holding part holding the tube and a connection end part attached to the incubator, wherein
the tube support unit is switchable between a first configuration in which the tube is supported such that the tube extends in a first direction from the first connection wall toward the second connection wall and a second configuration in which the tube is held such that the tube extends in a second direction intersecting with the first direction, and
the second configuration is switched to the first configuration by means of a restoring force of the tube support unit.

5. A cell culture device comprising:
an isolator having a glove and a first connection wall and accommodating a first container;
an incubator connected to the isolator via a second connection wall facing the first connection wall and accommodating a second container;
a flexible tube having one end connected to the first container and the other end connected to the second container and extending from the incubator to the isolator via holes respectively provided in the first connection wall and the second connection wall; and
a tube support unit having a holding part holding the tube and a connection end part attached to the incubator, wherein
the tube support unit is switchable between a first configuration in which the tube is supported such that the tube extends in a first direction from the first connection wall toward the second connection wall and a second configuration in which the tube is held such that the tube extends in a second direction intersecting with the first direction, and
the second configuration is switched to the first configuration by means of a restoring force of the tube support unit.
